(19) United States
(12) Patent Application Publication
(10) Pub. No.: US 2024/0058649 A1
(43) Pub. Date: Feb. 22, 2024

(54) METHOD AND APPARATUS FOR FAST LOW ENERGY RUNNING/TRAINING

(71) Applicant: eSens, Inc., Akron, OH (US)

(72) Inventors: R. Thomas SWIGER, Hudson, OH (US); Afrin Jahan LOPA, Northfield, OH (US)

(21) Appl. No.: 18/366,794

(22) Filed: Aug. 8, 2023

Related U.S. Application Data
(60) Provisional application No. 63/371,654, filed on Aug. 17, 2022.

(51) Int. Cl. A63B 24/00; A43B 3/38; A43B 3/44; A63B 69/00

(52) U.S. Cl. CPC A63B 24/0062; A43B 3/38; A43B 3/44; A63B 69/0035; A63B 2024/0068; A63B 2214/00; A63B 2220/22; A63B 2220/36; A63B 2220/40; A63B 2220/62

(57) ABSTRACT

A method for training a runner to reduce variation in a runner's step length while the runner runs is described herein, including positioning at least three markers, wherein a first marker is about a first distance from a second marker, the second marker is about the first distance from a third marker, and the second marker is about the first distance from the first and third markers, and instructing a runner to run and step on or near the markers while running.

### I. CROSS-REFERENCE TO RELATED APPLICATION

This application claims the benefit of U.S. Provisional Patent Application No. 63/371,654, filed Aug. 17, 2022, which is incorporated by reference herein in its entirety.

### II. BACKGROUND

The present teaching is directed to a method of improving running performance. More specifically, the present teaching is directed to the use of a sensor array, including a pressure sensor and accelerometer; a running physics model, to analyze stride-by-stride and step-by-step metrics; and a training method to improve a runners performance using said metrics.

Running as a sport and means of exercise continues to gain popularity due to its low barrier to entry for new runners. Despite this low barrier, proper running form and technique can take runners a long time to develop and even professional runners strive to find ways to improve their performance. With advances in wearable technology, many devices have been developed to instruct new or seasoned runners on how they can modify their performance to run more efficiently, reduce the risk of injury, reduce fatigue, increase speed, amongst other improvements.

While many devices aid runners in improving their performance there are still many areas in which the current technology does not provide a means of addressing specific issues with a runner's speed mechanics. Running performance is usually measured by maximum oxygen uptake (V·O2max), the lactate threshold and running economy. All these parameters usually are measured in a lab setting on a treadmill. This disclosure allows data to be collected when a runner runs either inside or outside and this disclosure measures running performance based on momentum change and Energy utilized Per Foot (E/ft) and Per Second (E/sec) during an entire run.

### III. SUMMARY

This summary is provided to introduce a selection of concepts in a simplified form that are further described below in the Detailed Description. This summary is not intended to identify key factors or essential features of the claimed subject matter, nor is it intended to be used to limit the scope of the claimed subject matter.

A method for training a runner to reduce energy consumption and increase speed utilizing a mobile monitoring device and running physics model is described in the present teaching.

A mobile monitoring device allows for the relevant metrics to be recorded at any location the runner would prefer to train instead of confined to a treadmill. The mobile monitoring device comprises a set of pressure sensors attached to a shoe insert which can be placed underneath a standard running shoe's insole. These pressure sensors provide information regarding the order and relative force with which different parts of a runner's foot strike the ground which can be used to inform the runner if they are overstriding. Additionally, the pressure sensors provide information regarding when a runner's foot strikes the ground allowing the timing of individual steps or strides. This information is processed within a housing which can attach to the outside of a runner's shoe. Within the housing is a printed circuit board (PCB) outfitted with an accelerometer and processor connected to the pressure sensors through conducting wire. The accelerometer measures g-force in the x, y, and z directions of the runner's foot throughout a given run. When processed with the pressure sensor data, the accelerometer data allows for the calculation through the running physics model of the energy expended by the runner during a given step.

The running physics model described in the present teaching is able to calculate the energy expended by a runner during any given step of a run using the information gathered by the mobile monitoring device. Using the standard Newtonian equation for Force and Energy, along with two kinematic equations, the physics model calculates the energy of a step. These equations are Force=Runner's Mass*Acceleration, Energy=Work=Force*Distance, Acceleration=ΔVelocity/ΔTime, and ΔDisplacement=(Initial Velocity+Final Velocity)*ΔTime/2. From these equations the following equation is derived for energy; Kinetic Energy=Work=0.5*Mass*(Final Velocity\hat{ }2−Initial Velocity\hat{ }2). This equation relies on the mass of the runner, and the difference in torso velocity between the foot landing, initial velocity, and leaving the ground, final velocity. The physics model calculates these velocities. Since no velocity change can occur between the first and second foot touching the ground, as the runner is in the air, the initial velocity of the runner will be the same as the final velocity of the runner when their next foot touches the ground. Hence, the final velocity of step N is the initial velocity of step N+1. This relationship lasts for the entire run. The initial velocity of step 1 is essentially zero because the runner is starting from rest.

The training method described in the present teaching is able to train a runner to expend less energy while running at any speed but particularly expend less energy while also increasing their speed. Using the running physics model described in the present teaching the training method recognizes that many runners have a significant variance in their speed between steps, accelerating and decelerating alternatively. This variance forces runners to have higher accelerations more often than they would need if they were more consistent and had smaller decelerations. It has been found that step acceleration variation is directly correlated to step length variation. The training method improves this consistency by training the runner's muscle memory to place their steps at a consistent length by running continually over a distance marked, or indicated by other consistent distance indicators, in increments equivalent to the determined optimal step distance for the runner. As acceleration and step length variation are directly correlated by improving step length consistency the runner will reduce momentum change variation. Once a runner has step length consistency the amount of energy, they are expending at any given speed is reduced. With step length consistency achieved the training method adds a metronome or other consistent timing feedback at the cadence (steps/minute) the runner is seeking to achieve. This trains the runner to place their steps at the consistent step length and time to achieve their desired speed while reducing energy consumption even further.

Still other benefits and advantages of the present subject matter will become apparent to those skilled in the art to which it pertains upon a reading and understanding of the following detailed specification.

### IV. Brief Description of the Drawings

The disclosure may take physical form in certain parts and arrangement of parts, aspects of which will be described in detail in this specification and illustrated in the accompanying drawings which form a part hereof and wherein:

FIG. 1 show a monitoring device such as that disclosed by the present teaching;

FIG. 2 shows ground strike waveforms and stride components of a runner's stride;

FIG. 2A shows a breakdown of an individual step during a run from a physics point of view;

FIG. 3 shows the V_{f}-Vᵢ value or momentum change of every step for a 3200 m run;

FIG. 4 shows the values measured and calculated using the monitoring system of the present teaching and the physics model of the present teaching;

FIG. 5 shows the positive momentum change (+Vf-Vi) values for a 400 m lap of a collegiate runner;

FIG. 6 shows the actual energy consumed for the same 400 m lap of FIG. 5 of the collegiate runner;

FIG. 7 shows the impact that momentum change has on energy consumed at a specific speed (initial velocity);

FIG. 8 shows the momentum change when the positive (Vf-Vi) values in FIG. 6 are reduced by 30%;

FIG. 9 shows the impact that the 30% reduction in momentum change shown in FIG. 8 can have on energy consumption.

FIG. 10 shows the variation of step length and step time of a collegiate runner for a 3200 m run;

FIG. 11 shows the step velocity and step length of a collegiate runner during a 400 m lap;

FIG. 12 shows an indoor running track that was used with the training method disclosed within the present teaching;

FIG. 13 shows the step time and step length for an untrained runner using the track shown in FIG. 12 without using the marks as a guide for step length;

FIG. 14 shows the energy consumed by the same runner for the run shown in FIG. 13;

FIG. 15 shows the step time and step length for said runner of FIG. 11 while running at a consistent step length using the three-foot guide marks shown in FIG. 12;

FIG. 16 shows the energy consumed by the same runner for the run shown in FIG. 15;

FIG. 17 shows data from laps 5 and 7 from the speed profile run of a trained collegiate runner-runner 2;

FIG. 18 shows the step length distribution for lap 7 of the speed profile run of runner 2;

FIG. 19 shows the step time and step length for lap 7 of the speed profile run of runner 2;

FIG. 20 shows the step final velocity and step length for lap 7 of the speed profile run of runner 2;

FIG. 21 shows the momentum change (V_{f}-Vᵢ) vs. step number for lap 7 of the speed profile run of runner 2;

FIG. 22 shows the energy consumed vs. step number for lap 7 of the speed profile run of runner 2;

FIG. 23 shows the power used vs. step number for lap 7 of the speed profile run of runner 2;

FIG. 24 shows part of the 400 m track with step markers on lanes 7 and 8 for step lengths of 6.0 and 6.5 feet. Lane 7 was used by runners 2 and 3.

FIG. 25 shows the step time and length versus step number of runner 2's Interval Step Training (IST) run on the marked track shown in FIG. 23;

FIG. 26 shows the pivot velocity V_{f} and step length vs. step number of a runner 2 Interval Step Training (IST) run on the marked track shown in FIG. 23;

FIG. 27 shows the momentum change (V_{f}-Vᵢ) vs. step number of a runner 2 Interval Step Training (IST) run on the marked track shown in FIG. 23;

FIG. 28 shows the energy consumed vs. step number of a runner 2 Interval Step Training (IST) run on the marked track shown in FIG. 23;

FIG. 29 shows the step length distribution of a runner 2 Interval Step Training (IST) run on the marked track shown in FIG. 23;

FIG. 30 shows a table comparing the lap 5 and 7 speed profile data of runner 2 with data from the Interval Step Training (IST) run on a marked track shown in FIG. 23;

FIG. 31 shows data from laps 5 and 7 from the speed profile run of a trained collegiate runner-runner 3;

FIG. 32 shows the step length distribution for lap 7 of the speed profile run of runner 3;

FIG. 33 shows the step time and step length for lap 7 of the speed profile run of runner 3;

FIG. 34 shows the step final velocity and step length for lap 7 of the speed profile run of runner 3;

FIG. 35 shows the momentum change (V_{f}-Vᵢ) vs. step number for lap 7 of the speed profile run of runner 3;

FIG. 36 shows the energy consumed vs. step number for lap 7 of the speed profile run of runner 3;

FIG. 37 shows the step time and length vs. step number of a runner 3 Interval Step Training (IST) run on the marked track shown in FIG. 23;

FIG. 38 shows the pivot velocity V_{f} and step length vs. step number of runner 3's Interval Step Training (IST) run on the marked track shown in FIG. 23;

FIG. 39 shows the momentum change (V_{f}-Vᵢ) vs. step number of runner 3's Interval Step Training (IST) run on the marked track shown in FIG. 23;

FIG. 40 shows the energy consumed vs. step number of runner 3's Interval Step Training (IST) run on the marked track shown in FIG. 23;

FIG. 41 shows the step length distribution of runner 3's Interval Step Training (IST) run on the marked track shown in FIG. 23;

FIG. 42 shows a table comparing the lap 5 and 7 speed profile data of runner 3 with data from the Interval Step Training (IST) run on a marked track shown in FIG. 23;

FIG. 43 shows a light strip that may be used to train a runner in accordance with this teaching;

FIG. 44 shows another aspect of a light strip used to train a runner in accordance with this teaching, specifically, it shows Arduino-programmed individually addressable LED strip light for use with Interval Step Training (IST);

FIG. 45 shows a graph of running speed versus oxygen uptake of two athletes;

FIG. 46 shows a traditional chemistry run model;

FIG. 47 shows comparison of step velocity and energy spent per second for two runners;

FIG. 48 shows a physics run model;

FIG. 49 shows speed trial run data for momentum change;

FIG. 50 shows the tipping point in a speed trial run;

FIG. 51 shows What If scenarios of Runner 1 and Runner 2 if the positive momentum change is brought under the pivot point;

FIG. 52 shows standard deviations of momentum changes for Runner 1 and Runner 2's speed profile runs;

FIG. 53 shows Runner 4's energy reduction when running on treadmill with metronome compared to when running on treadmill with no metronome;

FIG. 54 shows comparison of momentum change with and without a metronome;

FIG. 55 shows comparison of energy with and without a metronome;

FIG. 56 shows comparison of step length with and without a metronome;

FIG. 57 shows a graph of step length and step time without a metronome; and

FIG. 58 shows a graph of step length and step time with a metronome;

FIG. 59 shows step time, step length, and step velocity of Runner 2 in freestyle run to check muscle memory;

FIG. 60 shows step time, step length, and step velocity of Runner 2 in IST run following the freestyle run;

FIG. 61 shows % coefficient of variation of step time and step length for speed profile lap 5, speed profile lap 7, freestyle run, and IST run of runner 2; and

FIG. 62 shows step length distribution of runner 2 in freestyle and IST run.

### V. DETAILED DESCRIPTION

Referring now to the drawings wherein the showings are for purposes of illustrating aspects of the disclosure only and not for purposes of limiting the same, and wherein like reference numerals are understood to refer to like components.

The present teaching describes a method of training a runner to reduce the energy that they consume when running while also increasing the average speed of their run; utilizing a monitoring system and device alongside a physics model which describes how an individual runs.

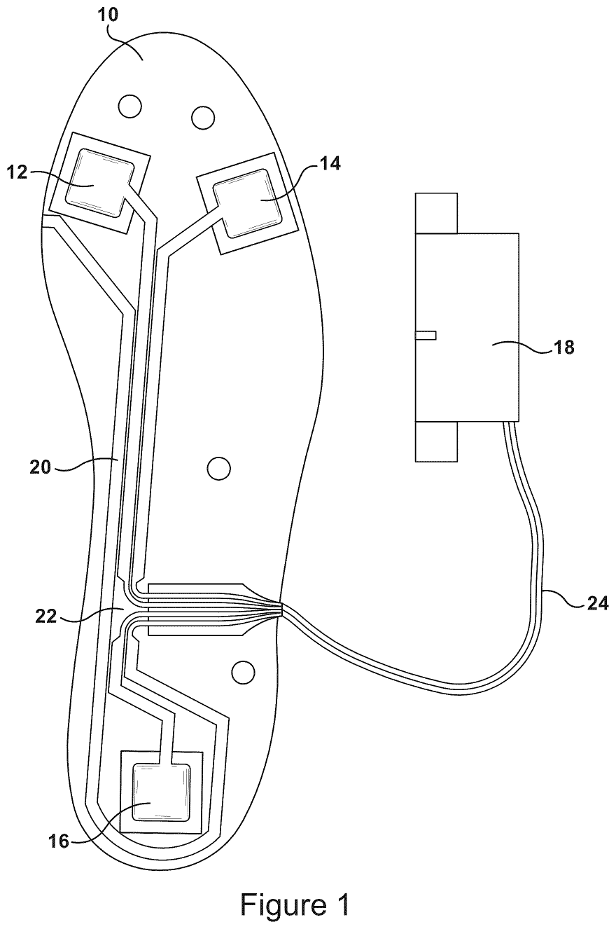

FIG. 1 shows a monitoring device comprising a shoe insert 10 that can be placed under the insole of a runner's running shoe and an electronics housing 18 which can clip onto a runner's running shoe. The shoe insert 10 can be put in either one shoe or both shoes. The shoe insert 10 includes a pressure sensor 12 placed at the interior forefoot of the shoe insert 10, a pressure sensor 14 placed at the exterior forefoot of the shoe insert 10, a pressure sensor 16 placed at the heel of the shoe insert 10, conducting material 20 routed to the lateral midfoot of the shoe insert 10, and conducting wires 22 connected to the conducting material 20 at the lateral midfoot of the shoe insert 10. The shoe insert 10 is connected via wires 24 to the electronics housing 18. The electronics housing 18 comprises an accelerometer 38 and processor 40 which processes the pressure sensor and accelerometer data. An accelerometer measures acceleration, orientation, and rotational velocity, but only the acceleration is used for determining the step-by-step metrics. The processed data is able to be uploaded to a computer or cloud-based platform that analyzes this data and inputs it into a physics running model. In one aspect of the present teaching, the accelerometer 38 is a nine-axis device that can measure time between strides. So, two types of data are collected by the system as a function time: voltage by the pressure sensors and G-force by the accelerometer. The electronic housing 18 can be used alone. The sensor array 10 and connecting wires 24 can be left out if ground strike waveform is not required.

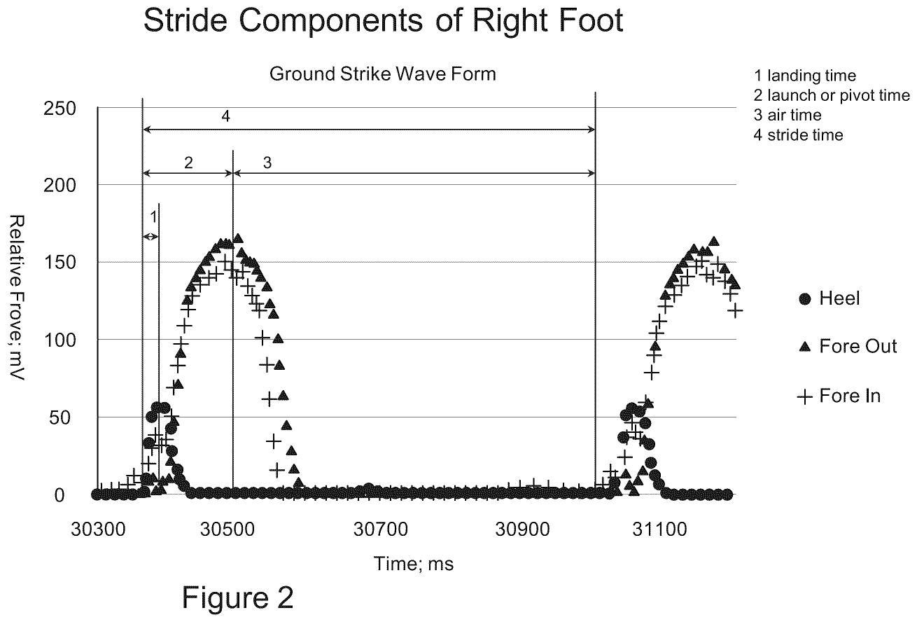

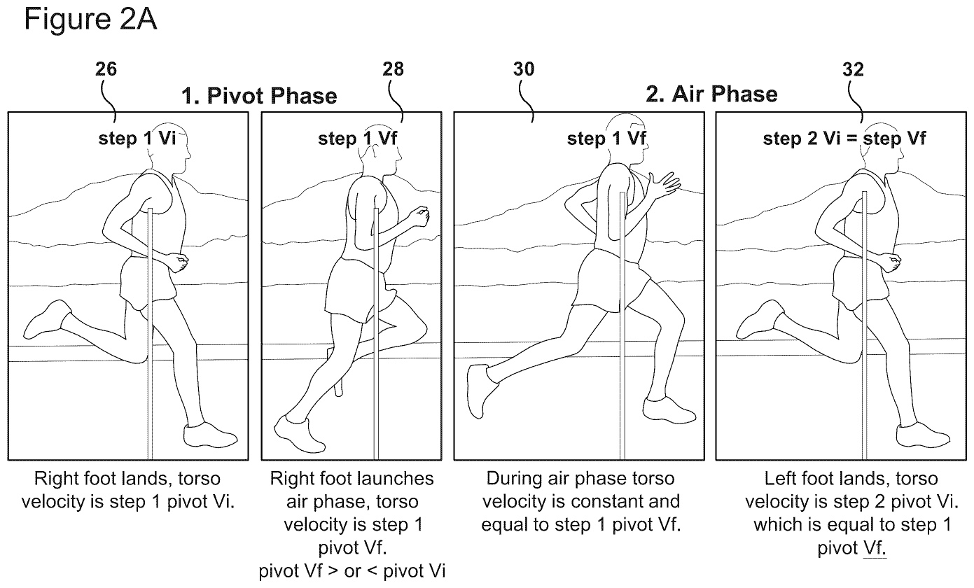

FIG. 2 shows the foot strike waveforms of a runner's stride. Based on the relative force applied on the sensors by the runner, the stride components can be determined. The foot strike waveform also shows whether a runner is a heel striker or forefoot striker. A collegiate distance runner athlete discovered that she is a heel striker using this eSens monitoring device. After training to change her foot strike pattern to be a forefoot striker, her running improved significantly.

FIG. 2A shows a runner at four distinct points during a step while running. In the physics model described in this disclosure the focus of the movement is on the runner's torso and on two phases in a running step—a pivot phase and an air phase. In the pivot phase, when the first foot lands 26, the torso has an initial pivot velocity Vᵢ defined for the entire step shown in FIG. 2. After this landing the foot launches 28 the runner into the air phase and the torso velocity at this launch 28 is defined as the final pivot velocity V_{f} for the entire step shown in FIG. 2. Once the runner is in the air 30 speed cannot be generated until the next pivot phase which begins when the second foot lands 32.

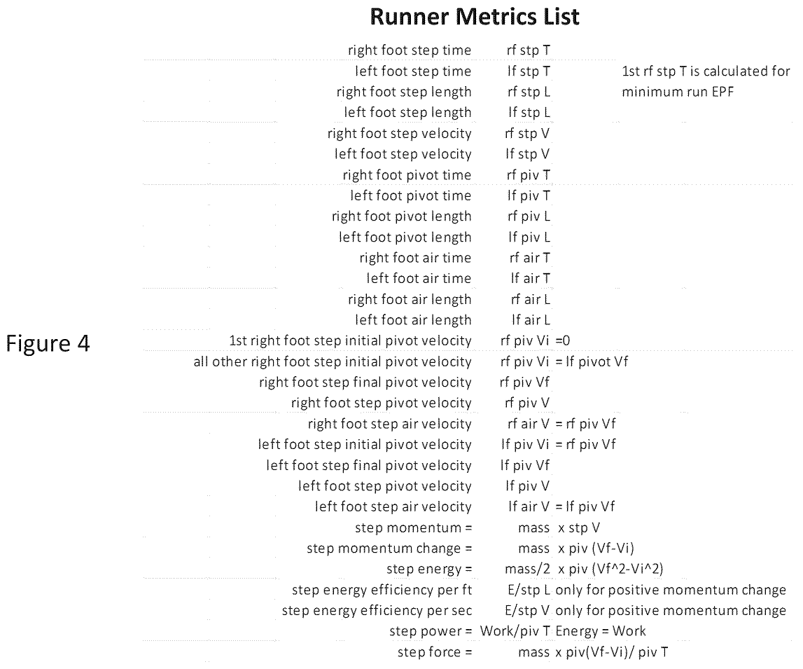

Pressure sensor data and accelerometer g-Force data are collected at a rate of 200 Hz and provide the physics running model with the landing and launch times of the runner along with the length of each stride. The physics running model then converts this data into all the runner metrics listed in FIG. 4. Step landing time is defined as the time the foot first strikes the ground, and step launch time is defined as the time the foot leaves the ground. Two accelerometers can be used at the same time by outfitting one accelerometer on each foot to have a common timeline and measure step-by-step metrics.

During the air phase 30, the velocity of the torso is constant and equal to FIG. 2's final pivot velocity, V_{f}. When the runner lands on the second foot 32 the initial pivot velocity Vᵢ of their next step will equal FIG. 2A V_{f}. The link between steps (where Vᵢ of one step is equal to V_{f} of the previous step) is a relationship that continues for the entirety of the length of a run. If FIG. 2's Vᵢ is less than FIG. 2's V_{f} there is acceleration of the runner, and energy is consumed. The energy consumed by this acceleration can then be calculated by the equation: Energy=runner's mass*(Vf\hat{ }2−V\hat{ }2)/2

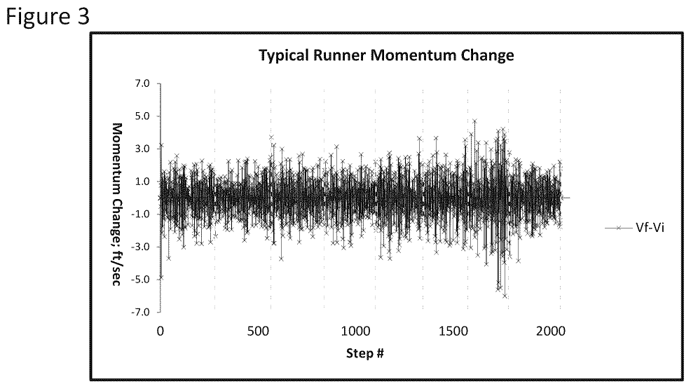

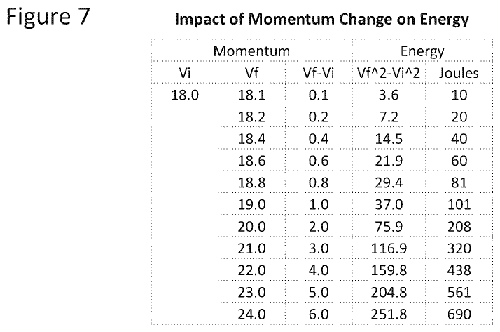

FIG. 3 shows the value of V_{f}-Vᵢ throughout an entire 3200 m run completed by a collegiate runner, runner 1. V_{f}-Vᵢ can be used as a step-by-step measure of the change in momentum of the runner. As FIG. 3 shows, the value of V_{f}-Vᵢ changes with every step of the 3200 m run over a relatively wide range. Almost 50% of the steps in FIG. 3 have positive V_{f}-Vᵢ values and are therefore representative of the runner accelerating. The other approximately 50% of steps in FIG. 3 have negative V_{f}-Vᵢ values and therefore are representative of the runner decelerating. Energy is only consumed when there is acceleration and is directly related to the value of (Vf\hat{ }2-Vi\hat{ }2)/2. Because of the difference in the relationship of momentum and energy with Vf and Vi, a small change in momentum can cause a large change in energy consumption as shown in FIG. 7. Therefore, if the momentum change of a runner can be reduced, energy consumption by that runner could be dramatically lowered.

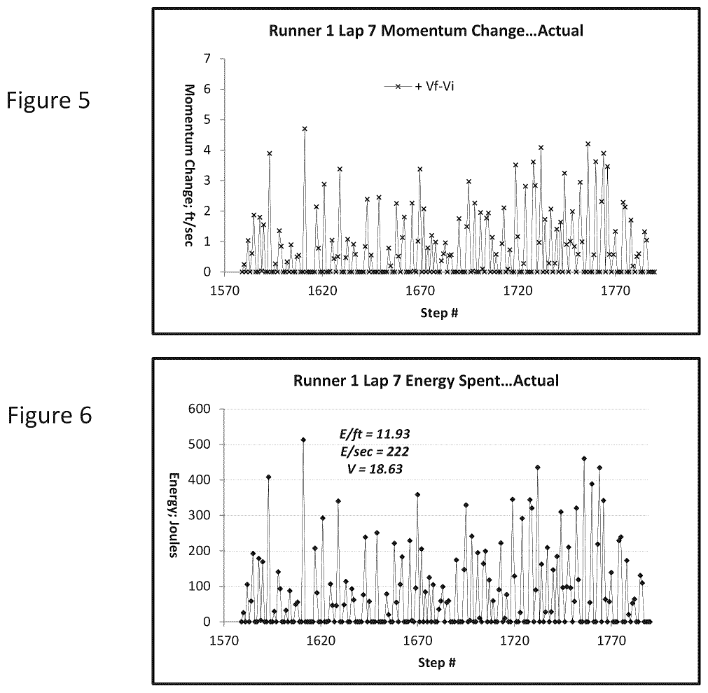

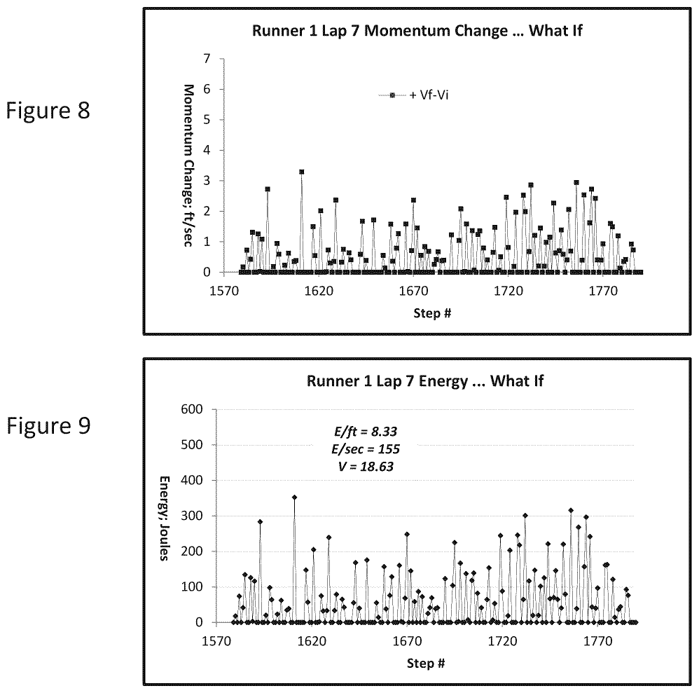

FIG. 5 shows the positive momentum changes for lap 7 (400 m) of a speed profile run of a collegiate runner. Since actual+(V_{f}-Vᵢ) values have considerable variation between acceleration steps the energy consumption associated with those steps is expected to be greater and is as shown in FIG. 6. If momentum change could be reduced by reducing the +(V_{f}-Vᵢ) values less energy would be consumed. FIG. 8 shows a hypothetical version of the run undertaken in FIG. 5 where the range of V_{f}-Vᵢ is reduced by 30%. In FIG. 9, a 30% reduction in energy occurs due to the consistency gained in V_{f}-Vᵢ. Despite this energy reduction the velocity of the runner remains the same. FIG. 7 shows that by reducing the range of V_{f}-Vᵢ values throughout a run a runner can use less energy and run at the same velocity.

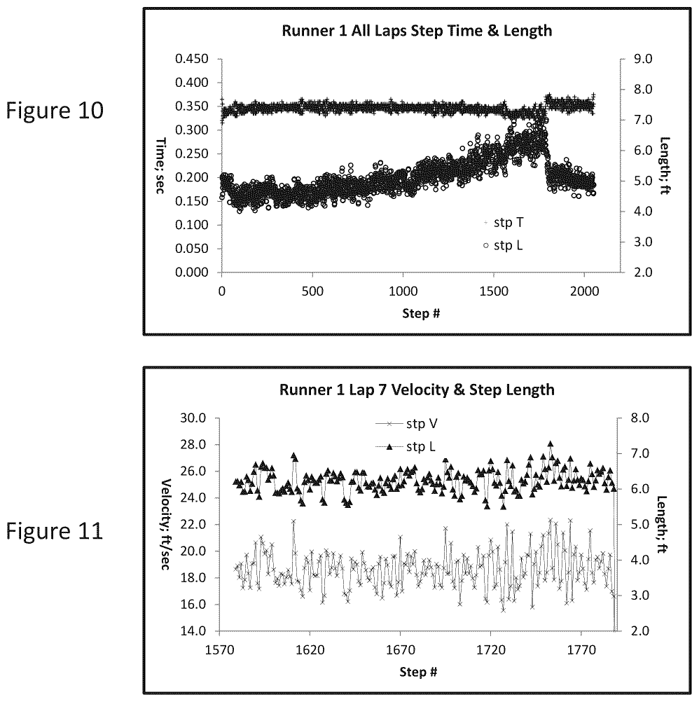

FIG. 10 shows the variation of step length and step time of a collegiate runner over a 3200 m run. FIG. 10 shows that this collegiate runner has a broad distribution in step length when compared to step time. FIG. 11 shows the step velocity and step length of a collegiate runner while running a 400 m lap. FIG. 11 shows that step length and step velocity are closely correlated such that by controlling the variation in the step length one should be able to reduce the magnitude of V_{f}-Vᵢ.

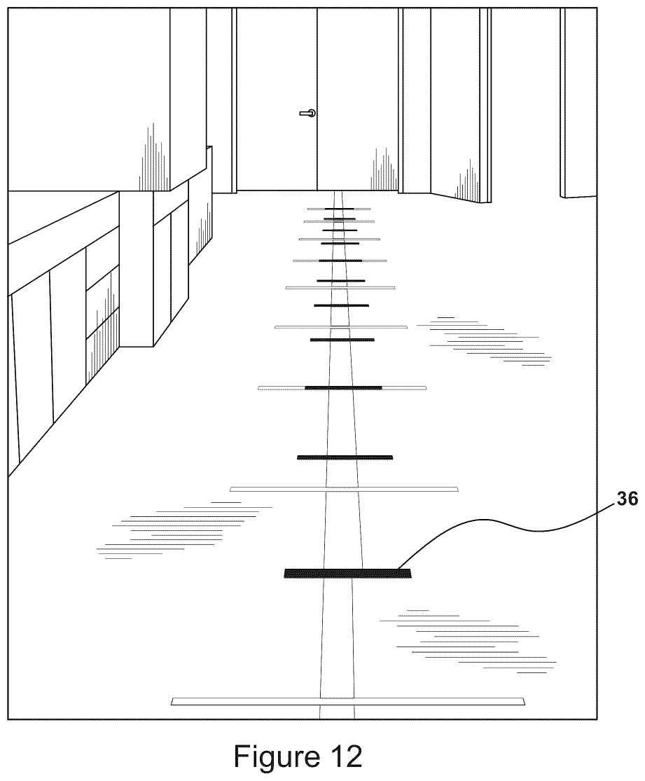

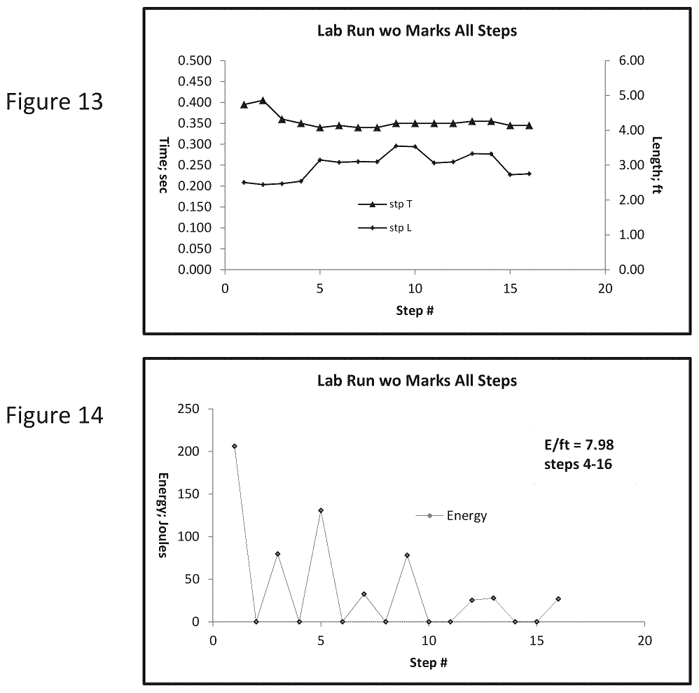

FIG. 12 shows a 50+ foot space which was used as a track for the training method disclosed in this teaching. As an example of the training method a runner with no training or prior running experience ran this track four times while wearing the monitoring system disclosed in this teaching. The average energy per foot of four runs was 7.67 joules/ft. FIG. 13 shows the step time and step length for the first run of four showing step length variation greater than the step time variation. FIG. 14 shows the energy profile of the first of the runs from FIG. 13 of this runner.

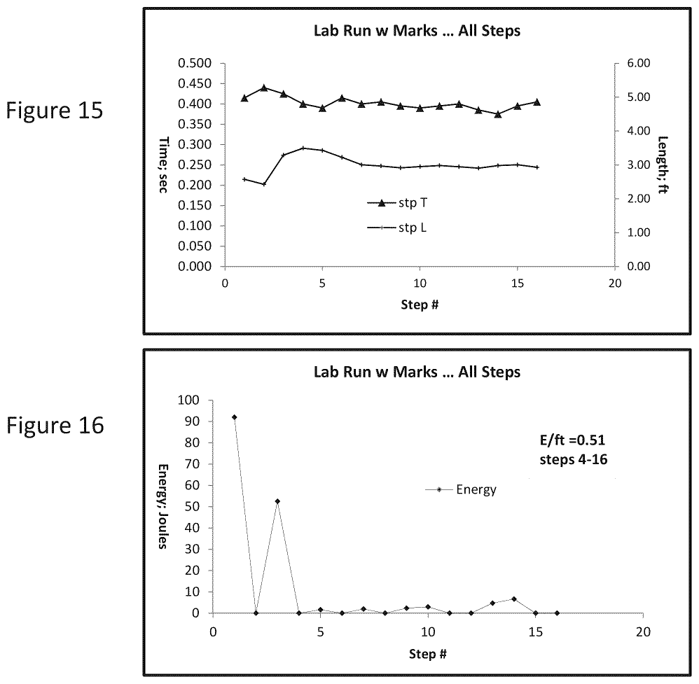

FIG. 12 additionally shows markers 36 at a consistent distance apart that was comfortable for the runner. The markers indicate where the runner should place their feet when running for a consistent step length. Step length can be indicated by physical markers or by sound, vibration, or any other sensory stimuli. Following the training method disclosed in the present teaching the runner practiced running the track and landing on the solid black marks 36 with each step to train the muscle memory of building a consistent step length. FIG. 15 shows the step length for the runner while running using the markers became much more consistent. FIG. 16 shows that the energy consumption during the run with the marks was reduced by as much as 82%. In order to run at higher speed with lower energy, the training with the marks can be practiced with a metronome or similar device to instruct the runner to strike the marks at a higher cadence. This allows the runner to run faster at lower energy.

To further verify the efficacy of the training method two collegiate distance runners—runner 2 and runner 3, completed a speed profile run. The runners completed eight laps on a 400 m outdoor track. In the first four laps, the runners gradually sped up to their tempo pace at which the 5th lap was completed. The 6th lap was targeted at a speed 10% faster than their tempo pace, and the 7th lap was targeted at a speed 25% faster than their tempo pace lap. The final lap is run as a recovery lap and so is completed at a slower pace.

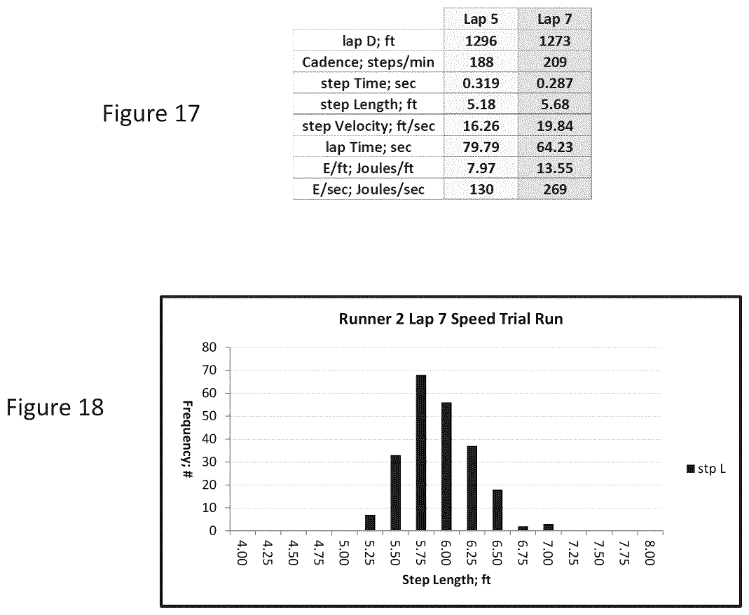

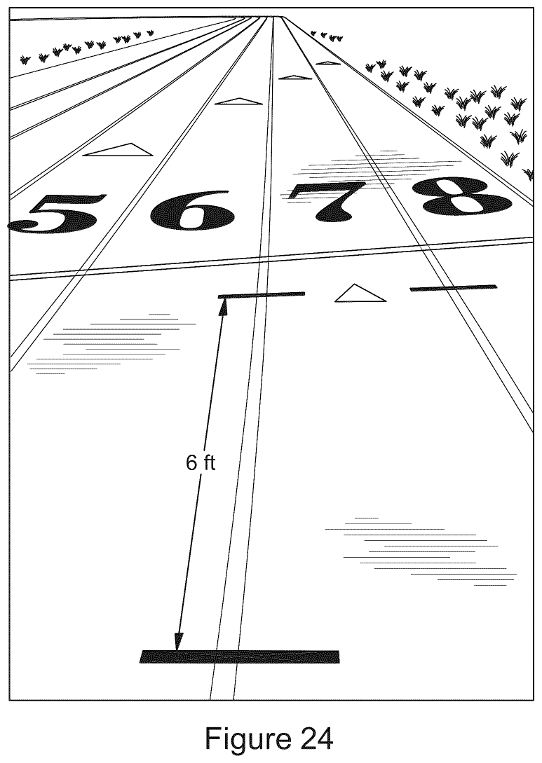

FIG. 17 provides a summary of runner 2's speed profile metrics for his tempo pace and fastest pace laps (Laps five and seven respectively). FIG. 18 shows runner 2's step length (step L) distribution for his fastest lap. Based on this information the runner's coach was able to determine that a step length of 6 ft was best for runner 2. Per the training method, at the same outdoor track, markers were set with an interval of 6 ft for 117 m plus a short start-up distance. After several practice runs, using the markers, the runner ran 117 m wearing the monitoring device taught in this disclosure and then the rest of the 400 m without markers at a recovery pace. Runner 2 continued the training run in the same pattern three more times. This training method is named Interval Step Training (IST) and the coach confirmed that IST fits well with the athlete's regular interval training. FIG. 24 shows the outdoor track with markers at 6.0 ft intervals at the lane line between lanes 6 and 7.

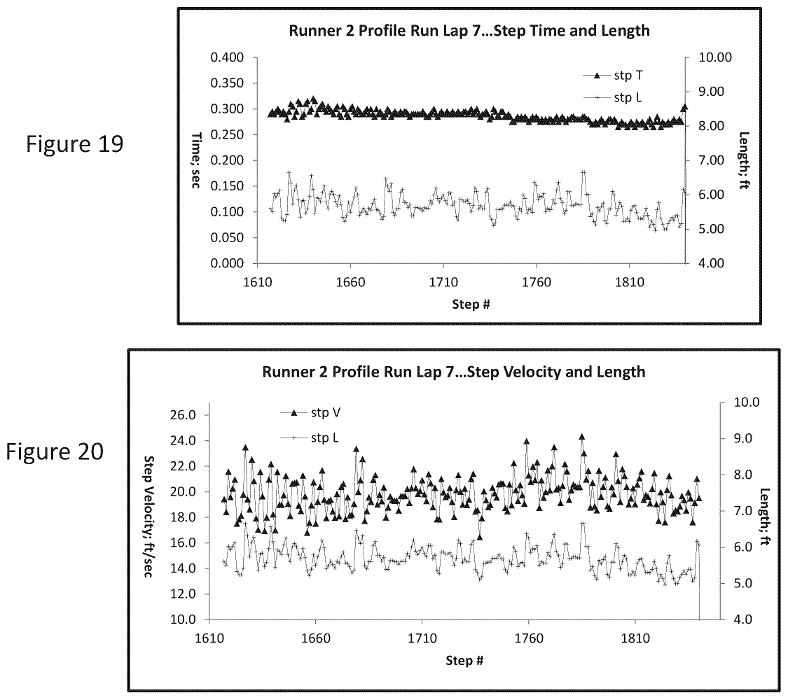

FIG. 19 shows the step time and step lengths for lap 7 of the runner 2's speed profile run. Step length changes at every step whereas the step time is more consistent throughout the lap. FIG. 20 shows the step velocity changes with step length changes.

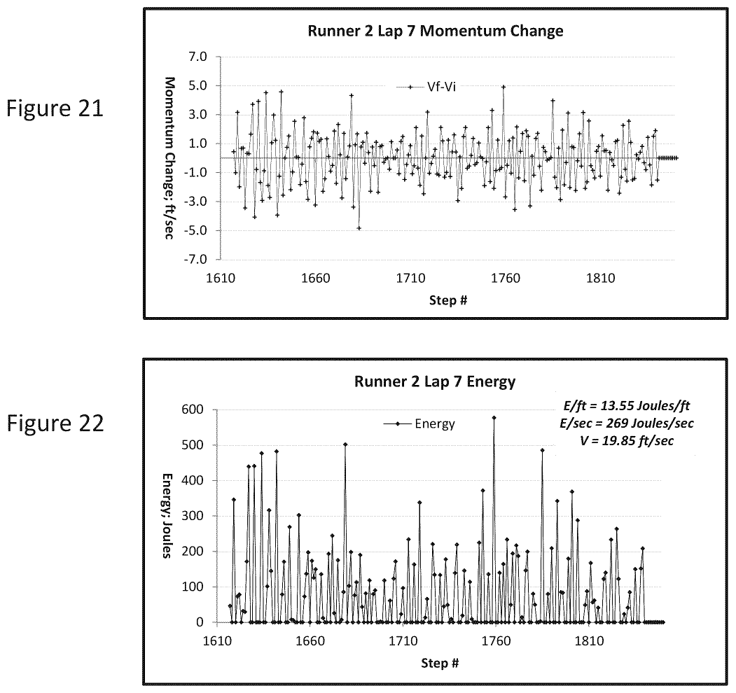

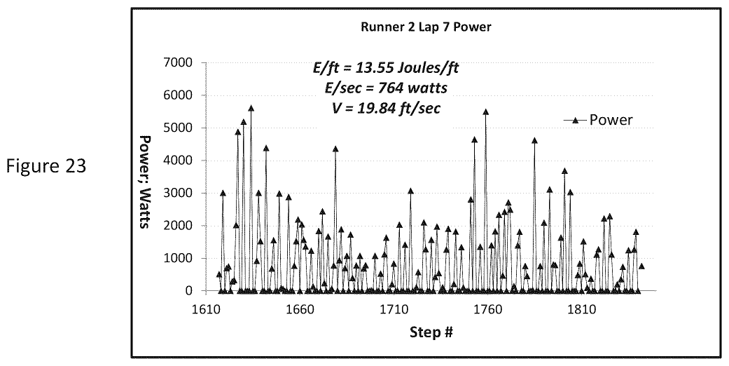

FIG. 21 shows the V_{f}-Vᵢ values for lap 7 of runner 2's speed profile run. FIG. 22 shows the energy consumed for the same lap 7 and FIG. 23 shows the power used for lap 7 which was calculated by dividing energy of the acceleration steps by the pivot time of that step. In this lap, 764 watts of power was used on average, 17,254 joules of energy in total for the lap, with an average speed of 19.84 ft/sec.

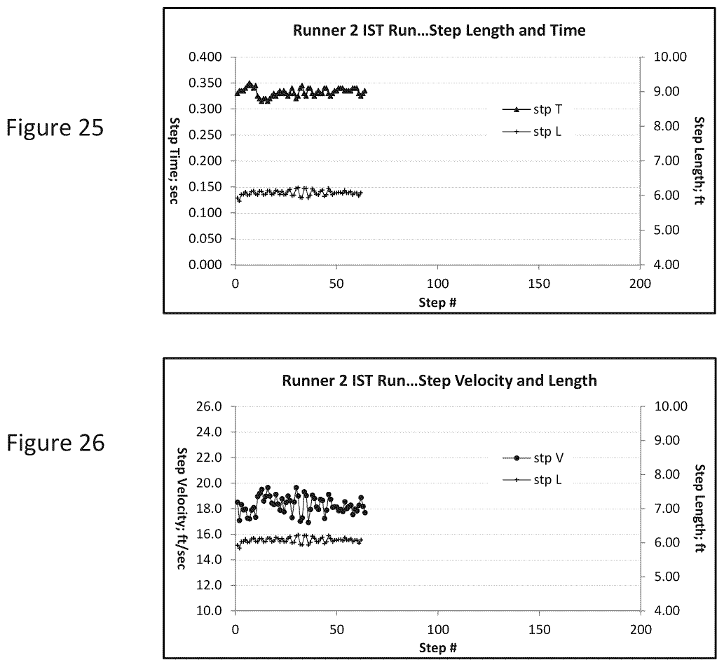

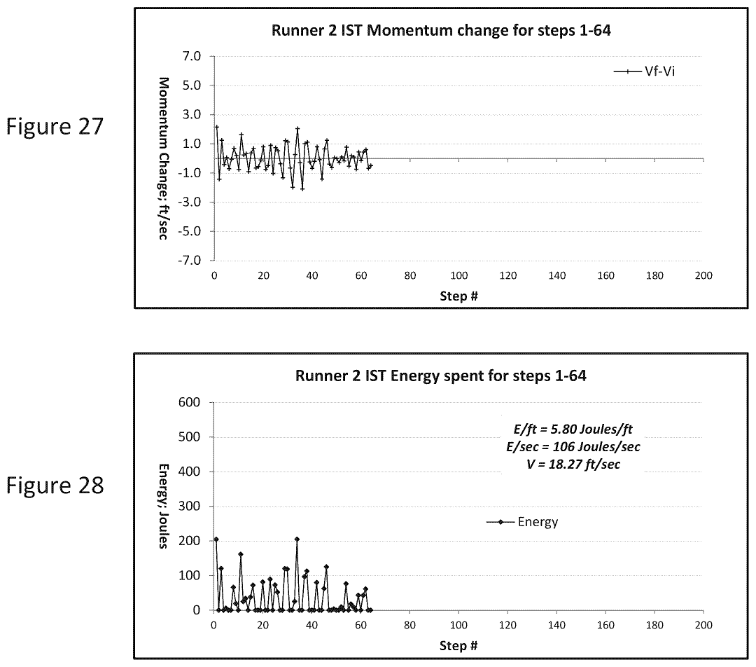

FIG. 25 shows the step times and lengths for the IST run with markers by runner 2. The step lengths show relatively small variations compared to the step lengths of lap 7 of speed profile run seen in FIG. 19. This is also evident in the momentum changes (V_{f}-Vᵢ) for the IST run shown in FIG. 27 and the energy consumed shown in FIG. 28.

FIG. 26 shows the step lengths and final velocities for the IST run with markers by runner 2. As with the step lengths, the step final velocity variations are also smaller compared to FIG. 20.

FIG. 27 shows V_{f}-Vᵢ values and FIG. 28 shows energy consumed for the IST run with markers by runner 2.

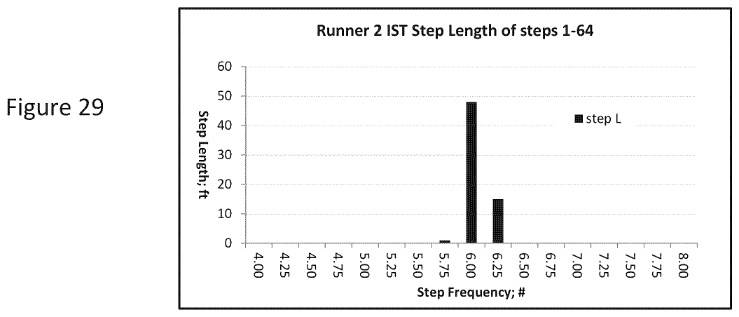

FIG. 29 shows the step length distribution for runner 2 in the run completed after using the IST method. FIG. 28 shows consistent step lengths of 6 ft (48 times out of 64 steps).

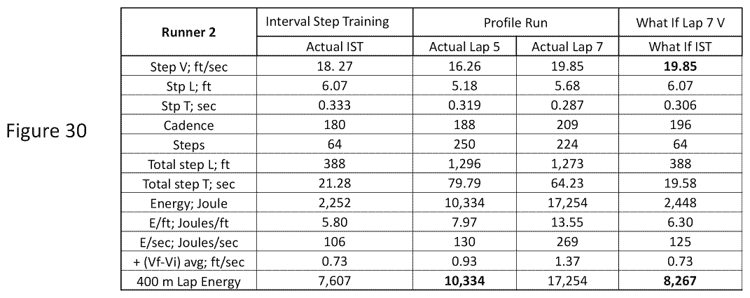

FIG. 30 compares a summary of the speed profile metrics for runner 2's tempo pace and fastest pace laps (laps five and seven respectively) with the IST run with step markers. The key comparison is the energy used in lap 5 (10,334 joules) with the projected energy used in a 400 m IST run (8,267 joules) at the Lap 7 velocity. The 8,267 joules are 26% lower than the 10,334 joules used in lap 5 profile run. Thus, it is plausible that by utilizing the IST method runner 2 can run faster at lower energy and lap 7 speed can become runner 2's new tempo pace.

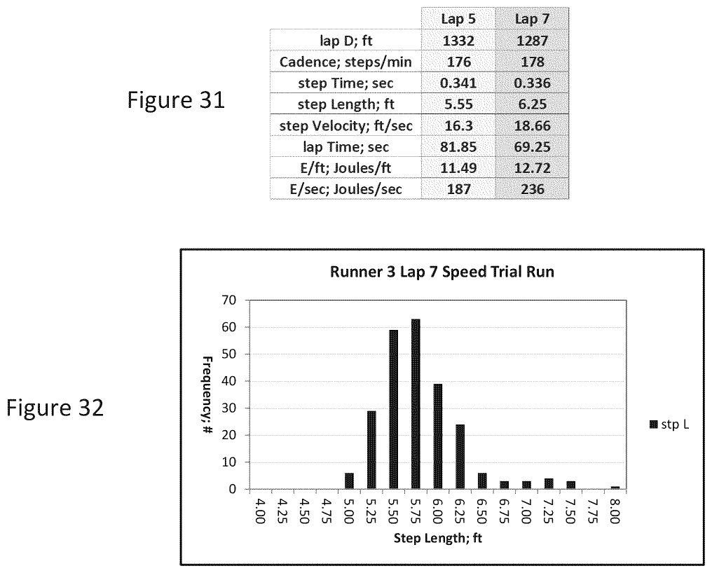

FIG. 31 provides a summary of runner 3's speed profile metrics for his tempo pace and fastest pace laps (laps five and seven respectively). FIG. 32 shows the runner 3's step length (step L) distribution for his fastest lap. Based on this information the runner's coach was able to determine that a step length of 6 ft for 117 m plus a short start-up distance. After several practice runs, using the markers, the runner ran 117 m wearing the monitoring device taught in this disclosure and then the rest of the 400 m without markers at a recovery pace. Runner 2 continued the training run in the same pattern three more times. This training method is named Interval Step Training (IST) and the coach confirmed that IST fits well with the athlete's regular interval training. FIG. 24 shows the outdoor track with markers at 6.0 ft intervals at the lane line between lanes 6 and 7.

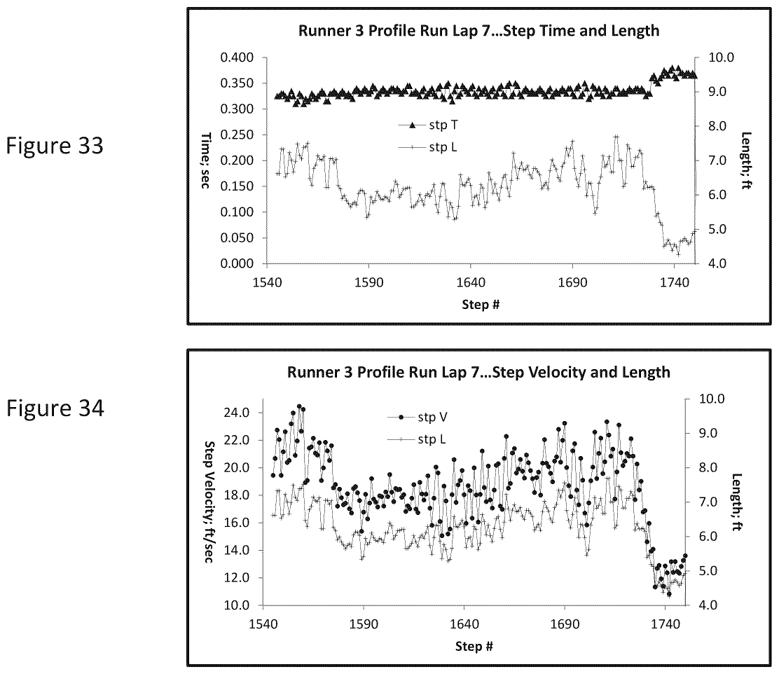

FIG. 33 shows the step time and step lengths for lap 7 of runner 3's speed profile run. Step length changes at every step whereas the step time is more consistent throughout the lap. FIG. 34 shows the step velocity changes with step length changes.

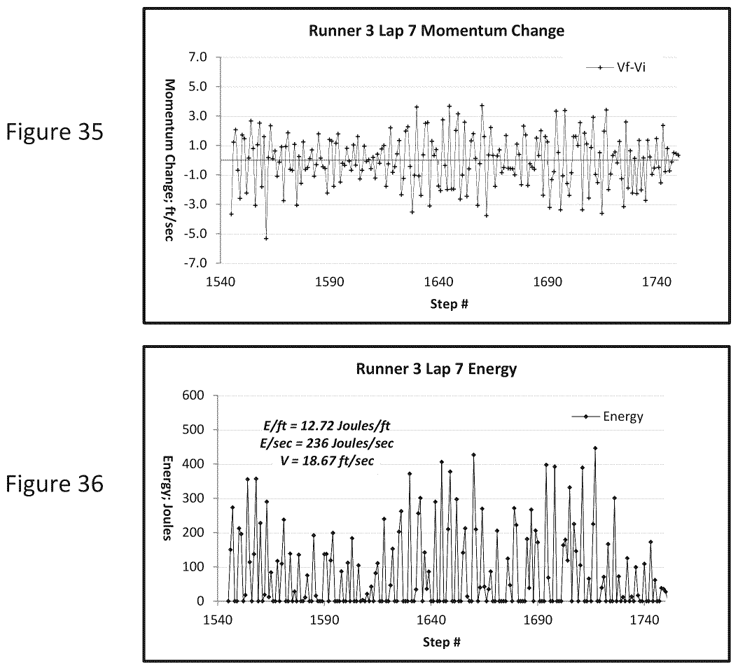

FIG. 35 shows the V_{f}-Vᵢ values for lap 7 of runner 3's speed profile run. FIG. 36 shows the energy consumed for the same lap 7. In this lap, energy was consumed at an average rate of 236 joules per second, 17,254 joules in total for the lap, with an average speed of 18.67 ft/sec.

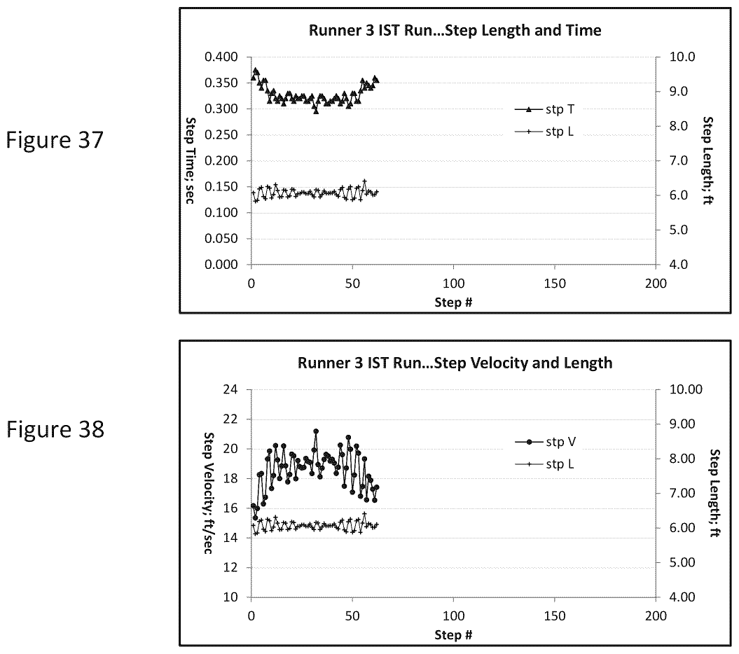

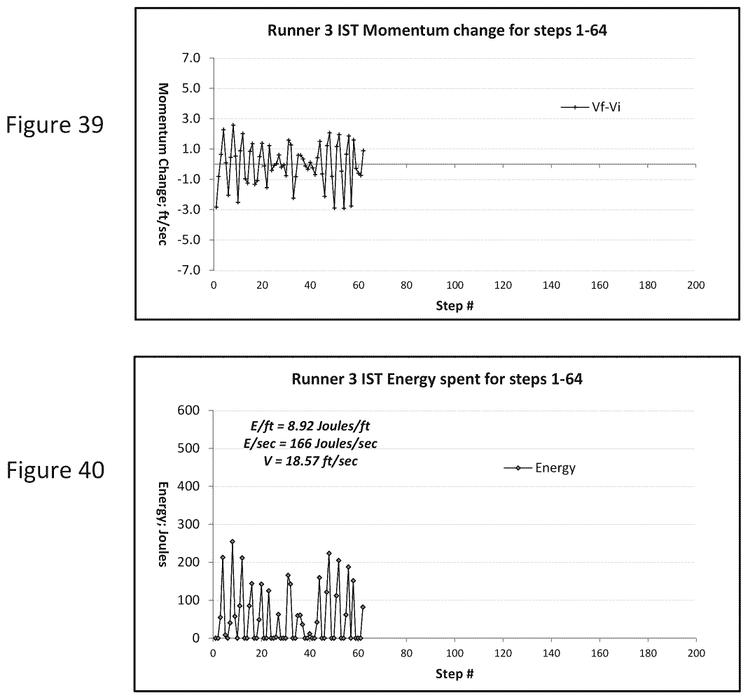

FIG. 37 shows the step times and lengths for IST run 2 with markers by runner 3. The step lengths show relatively small variations compared to the step lengths of lap 7 of speed profile run seen in FIG. 33. This is also evident in the momentum changes (V_{f}-Vᵢ) for the IST run shown in FIG. 39 and the energy consumed shown in FIG. 40.

FIG. 38 shows the step lengths and final velocities for the IST run with markers by runner 3. As with the step lengths, the step final velocity variations are also smaller compared to FIG. 34.

FIG. 39 shows V_{f}-Vᵢ values and FIG. 39 shows energy consumed for IST run with markers by runner 3.

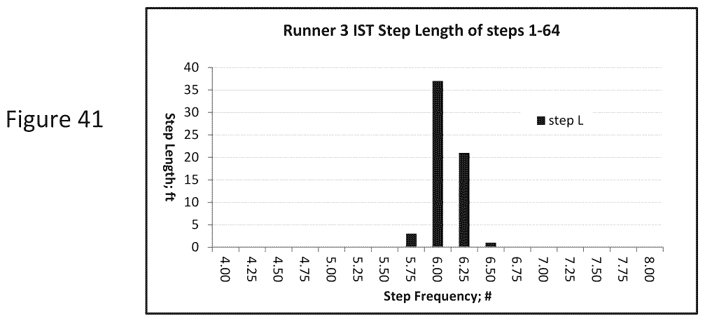

FIG. 41 shows the step length distribution for runner 3 in the run completed after using the IST method. FIG. 41 shows consistent step lengths of 6 ft (37 times out of 64 steps).

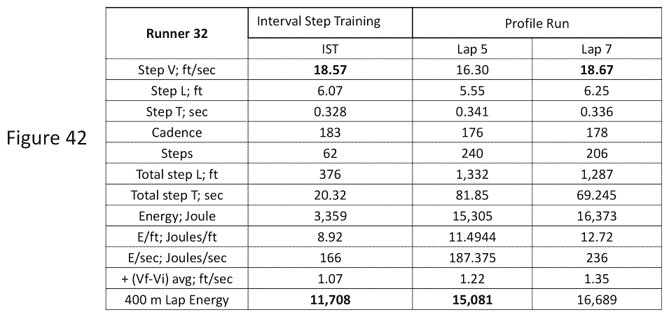

FIG. 42 compares a summary of the speed profile metrics for runner 3's tempo pace and fastest pace laps (laps five and seven respectively) with the IST run with step markers. The key comparison is the energy used in lap 5 (15,081 joules) with the projected energy used in a 400 m IST run (11,708 joules) at 99.5% of lap 7 velocity. The 11,708 joules are 22% lower than the 15,081 joules used in lap 5 profile run. Thus, it is plausible that by utilizing the IST method runner 3 can run faster at lower energy and lap 7 speed can become runner 3's new tempo pace.

Through using the training method disclosed in the present teaching, a runner can run at a faster speed over a longer distance using less energy. For example, a 25% faster run using 25% less energy. The monitoring system and physics model disclosed herein determine the force, energy, and power spent for every step allowing the runner to modify their speed mechanics to run more efficiently. The monitoring system disclosed herein provides runners detailed information on their foot strike pattern which can ultimately reduce the possibility of injury and help a runner run faster by training them to be fore-foot runners as opposed to heel striking runners. Coaches may also use the training method, monitoring system, and physics model disclosed herein to analyze their runners' results and analytics, and coach their runner, without physically being present with the runner. Recreational runners will also be able to access these teachings without the expenses normally associated with such services.

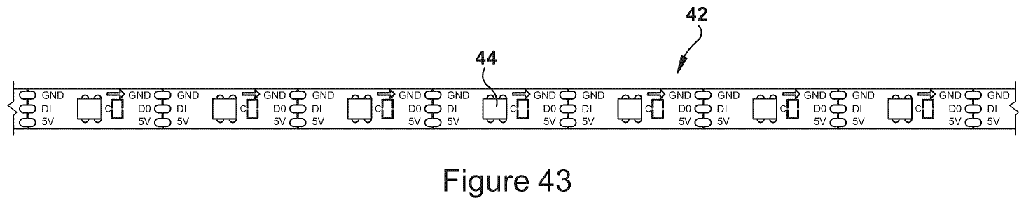

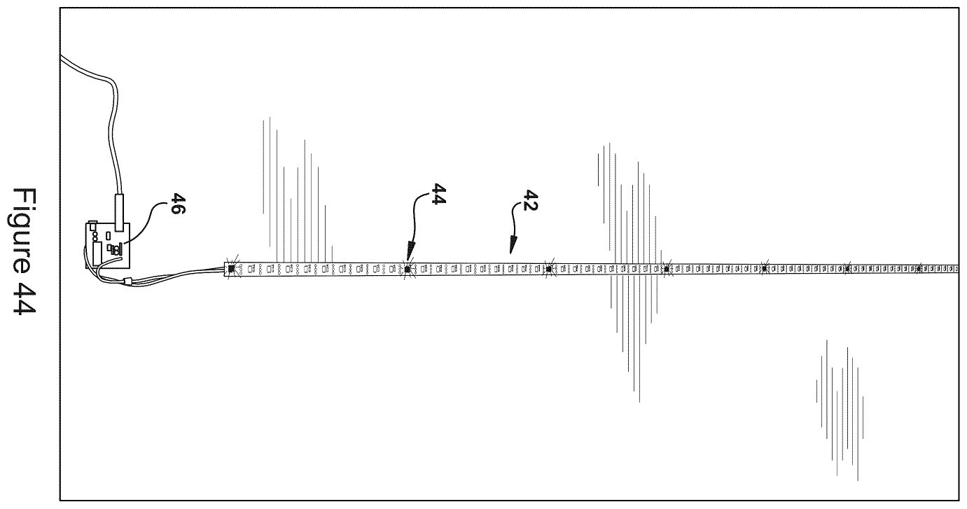

FIG. 43 shows a light strip 42 with a plurality of light 44. Light 44 may be a Light Emitting Diode (LED). Light strip 42 may comprise a strip of light. Individuals or groups of light 44 can light up along light strip 42. Light strip 42 may be controlled by a microprocessor 46 such as an Arduino microcontroller. Microprocessor 46 may be programmed by using a conventional computer and using open-source code. Light strip 42 and microcontroller 46 may be powered by an internal or external power supply 48.

A person can use light strip 42 instead of physical markers to indicate where a runner should step to have a certain step length. To do this, microprocessor 46 may be programmed to make one or more light 44 on light strip 42 illuminate that corresponds to where markers 36 would be. Then, light strip 42 is placed along a path that a runner will use to practice running at his or her earlier-determined step length. For example, light strip 42 could be placed in between two lanes on a track to run down the length of the track. Then, a runner can run at the desired step length by stepping near the light 44 that is lit on light strip 42 while running. Running consistently at the desired step length incorporates that step length into the muscle memory of a runner. Once incorporated, the runner will run faster using less energy.

For multiple runners to use the same step length marker system for Interval Step Training, eSens has developed a system of using light strip 42 and microprocessor 46, more specifically, a system of individually addressable LED strips, together with an Arduino UNO R3 microcontroller board. Multiple, waterproof, five-meter-long individually addressable LED strips are connected end to end to provide lighted markers at any desired interval for IST runs. The LED strips may be powered from a nearby outlet, available on most tracks for timing devices, or from a portable battery. The number of LED strips connected is dependent on the training run distance. Using the Arduino microcontroller, runners set the light interval to match their desired step length. Adafruit open-source code has been modified and uploaded to Arduino for this purpose. Runners set their desired light interval via their phone or laptop. As shown in FIG. 44, the LEDs turn on at intervals matching the input step length. Runners then use the bright LEDs as markers for their Interval Step Training. In addition to step length, the number of steps taken by the runner is also part of the present teaching. In order to set up the markers to ascertain information, in one aspect of the present teaching, the runner will take at least ten steps during a test trial. It is also to be understood that the number of steps can be between 10 and 80, between 20 and 70, between 30 and 60, and between 40 and 50. It is also to be understood that any number of steps over 80 could be used as well.

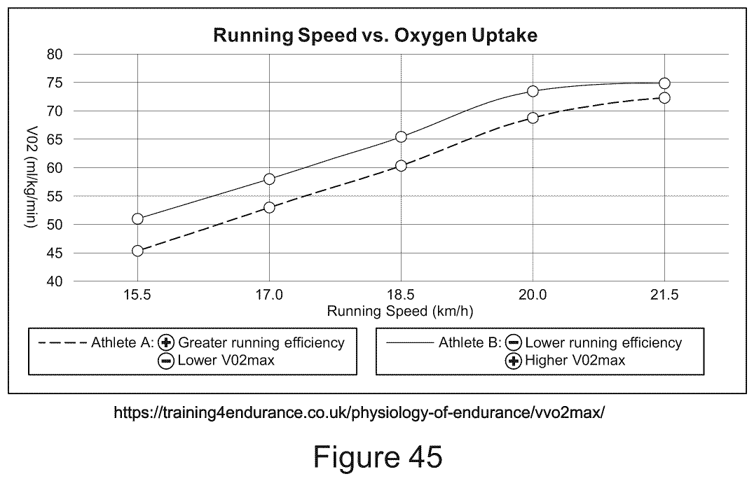

Research has shown that one of the strongest predictors of endurance running performance, across a broad range of distances, is the minimum velocity at which a runner achieves peak oxygen uptake, vVO₂max. The chart in FIG. 45 shows the oxygen uptake for two runners as a function of running speed. Each runner ran on a treadmill at the same range of speeds, and it was concluded that Athlete A had greater running efficiency because at each speed Athlete A used less oxygen, but since Athlete B's vVO₂ max was higher, Athlete B would be the better runner. Most collegiate coaches are aware of this vVO₂ max theory, but do not have the ability to determine their runner's vVO₂ max. However, since Interval Training (IT) has been shown to improve vVO₂ max, coaches tend to incorporate some form of IT into the training regimen of each of their runners.

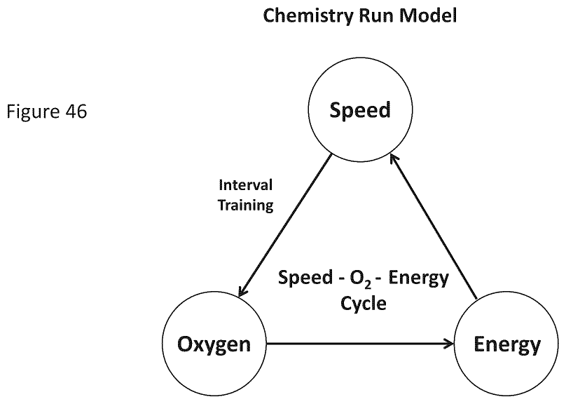

If the conclusions drawn in FIG. 45 are valid, then oxygen consumption of a runner is determined directly by running speed. Since oxygen consumption determines energy production via a series of known chemical reactions, energy consumption is also tied directly to runner speed. Therefore, as a runner runs faster, energy consumption must increase. These results suggest the Chemistry Run Model, shown in FIG. 46, as a closed loop system connecting runner speed generation to oxygen consumption/oxygen consumption to energy consumption/energy consumption to speed generation. If the Chemistry Run Model is correct, then one would expect Athletes A and B to have the same oxygen consumption at the speeds run. According to exercise physiologists these differences are likely accounted for by small physiological differences between runners.

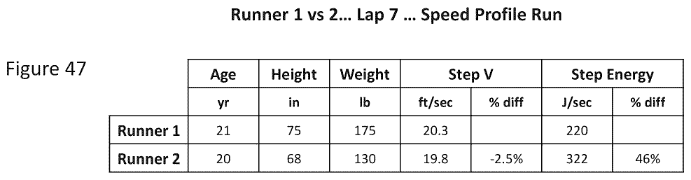

FIG. 47 show results for laps run on a track for collegiate runners 1 and 2. These runners were teammates, ran a speed profile run on the same day, on the same track, and had had essentially the same training. Runner 2 ran 2.5% slower than runner 1 in their fastest lap (lap 7) but consumed 46% more energy. These results were reviewed separately by two professors of exercise physiology and one former collegiate runner. Both concluded that it was highly unlikely that physiological differences between runners 1 and 2 could account for the difference in energy consumption.

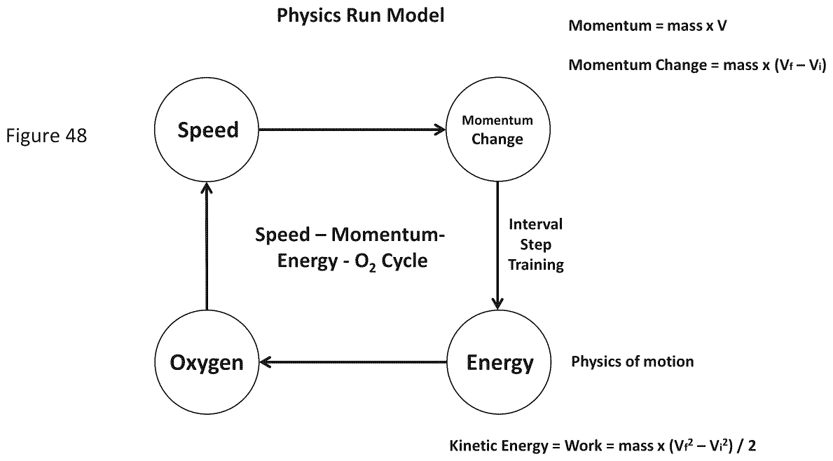

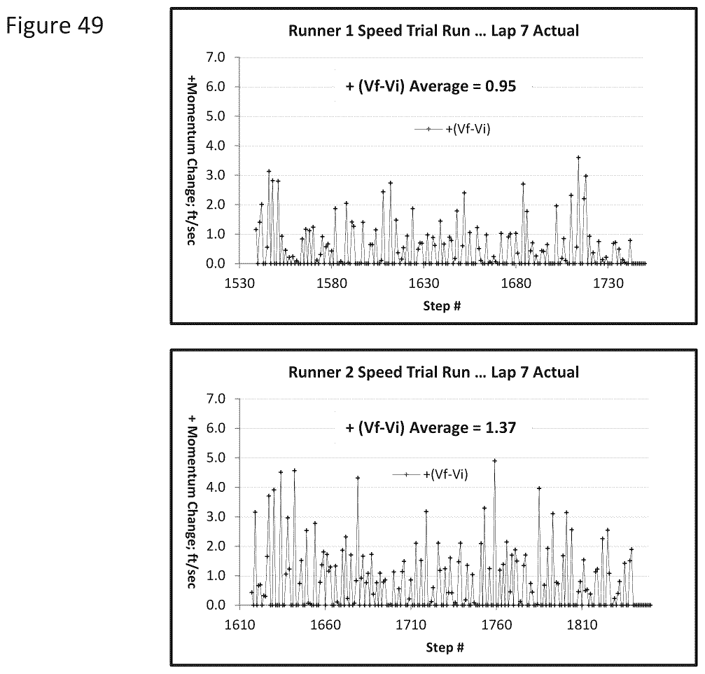

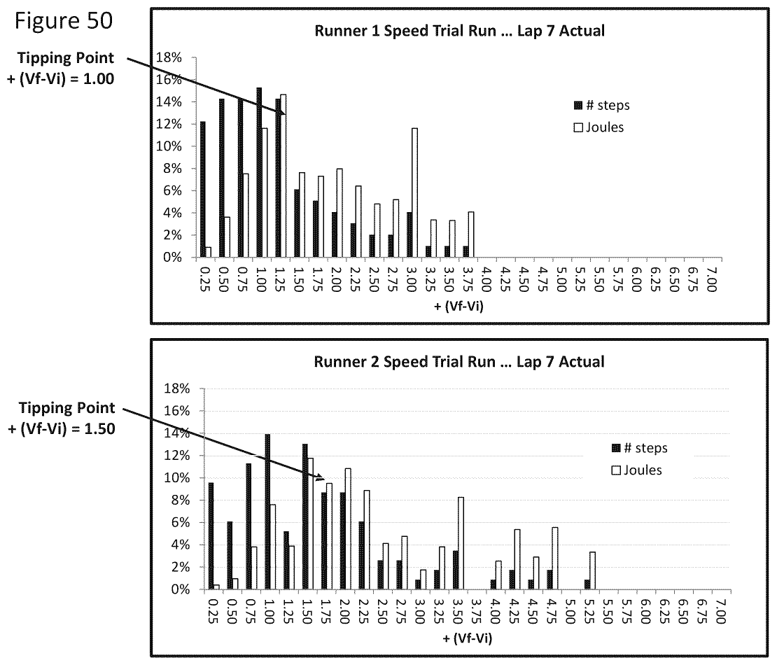

The Physics Run Model described in paragraphs 0078-0082 and shown in FIG. 48 can easily account for the results shown in FIG. 47. The Physics and Chemistry Run Models are both closed loops, but the inclusion of Momentum Change in the Physics Run Model and reversing the flow direction makes a significant difference. FIG. 49 shows the positive momentum changes in lap 7 for runners 1 and 2. The plots look somewhat similar, but they are very different from a performance perspective. The average momentum increase (+(Vf-Vi)) for runner 2 is 1.37 ft/sec but only 0.95 ft/sec for runner 1, a difference of 44%; similar to the 46% difference in lap energy. When the percentage of steps and energy are plotted, FIG. 50, as a function of the distribution of positive momentum change, the difference between the runners becomes even more apparent. The tipping point, defined as the maximum positive momentum change with step percentage greater than energy percentage, is 1.50 for runner 2 but 1.00 for runner 1.

The Physics Run Model data clearly supports the fact that Runner 1, although running 2.5% faster than runner 2 can consume far less energy than runner 2 by taking into account the momentum change of each runner over the course of the lap.

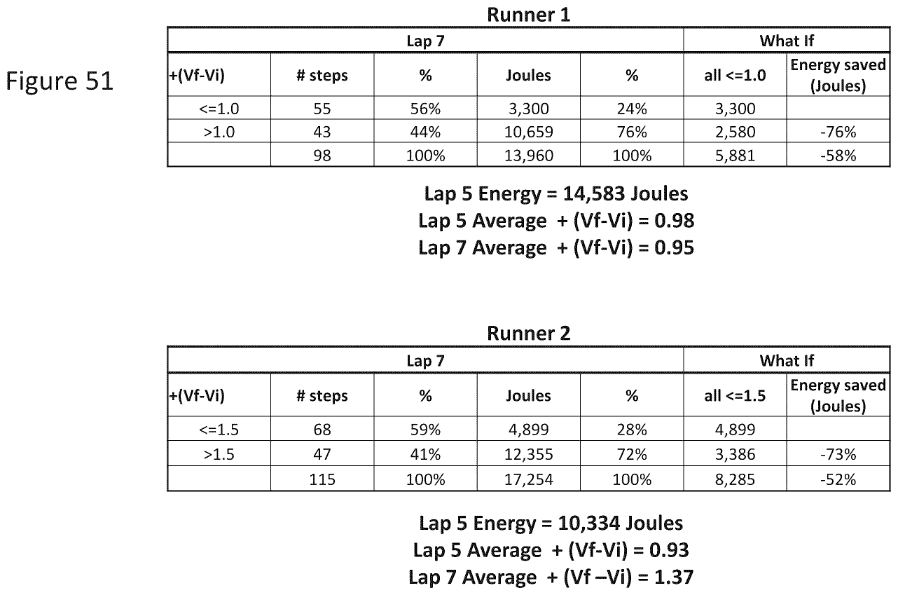

In addition, the “what If” scenarios shown in FIG. 51 for each runner show that each can significantly reduce their energy consumption by running each step at or below their tipping point. Thus, enabling higher overall lap run speed.

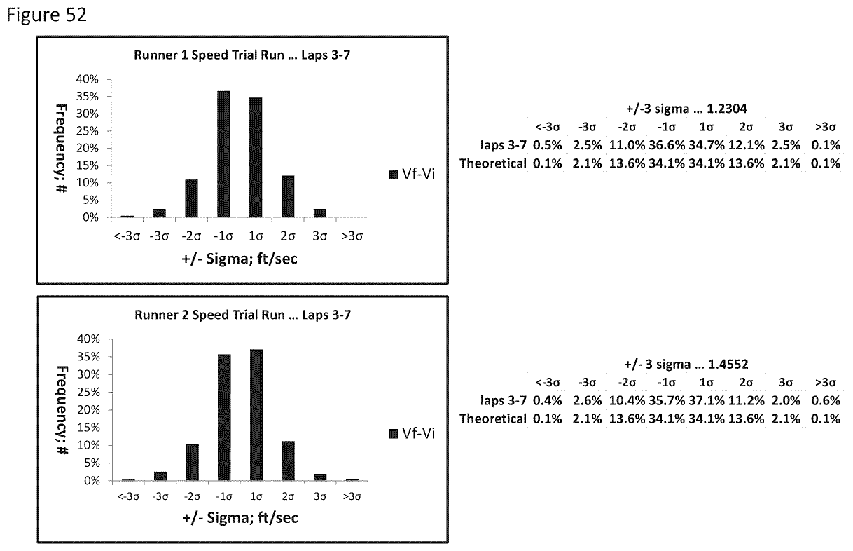

Another interesting aspect of these runs is the similarity in the distribution of momentum change, that is (V_{f}-Vᵢ). As shown in FIG. 52, although the standard deviation for runner 1 at 1.2304 is significantly smaller than the standard deviation for runner 2 at 1.4552, their actual+/−3 sigma distributions are quite similar, and both correlate well with theoretical values for a standard distribution. This result has been observed for other runners as well and leads to the thought that most, if not all, runners may naturally run with ever changing momentum.

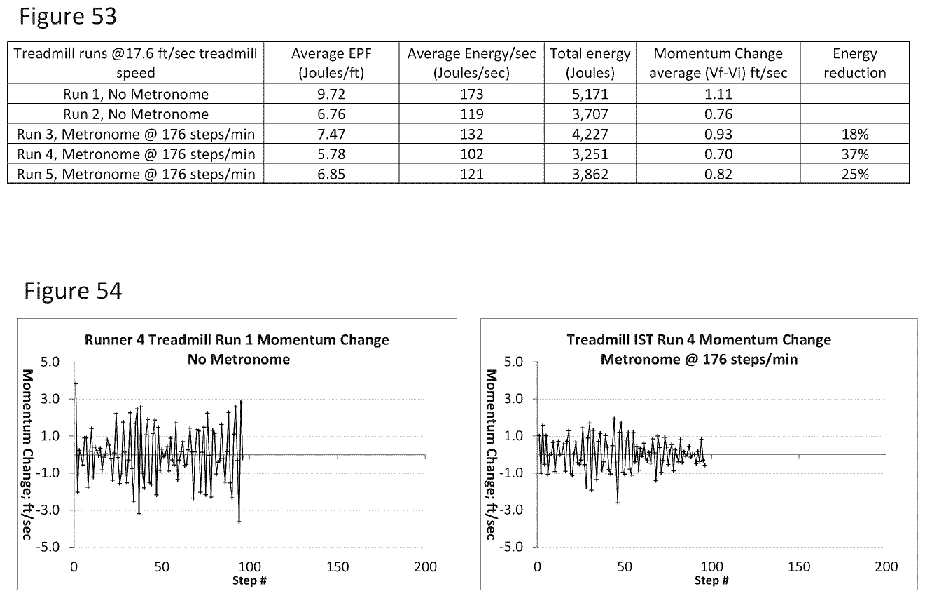

A treadmill can be used for Interval Step Training (IST) as shown in FIGS. 53-58. Runner 4's fastest speed profile lap was lap 7, with an average speed of 17.57 ft/sec. The step length distribution of this speed profile lap indicated that a step length of 6.0 feet would be ideal for IST purposes. Thus, the treadmill speed was set at 17.6 ft/sec with a target cadence for the runner of 176 steps/min. At this speed and cadence his step length would be 6 feet. To ensure the target cadence a metronome was used to provide an audible indicator as to when his foot should land on the treadmill for each step. After a 30 second IST run at 17.6 ft/sec with the metronome, the treadmill speed was reduced to 12 ft/sec, recovery speed, and the metronome silenced for 90 seconds at which time the next IST run was initiated. Five IST runs were completed using this procedure. IST runs 3, 4, and 5 were completed with the metronome turned on, and IST runs 1 and 2 were completed with the metronome turned off. Also, IST can be practiced on a treadmill with any incline or at an outdoor cross-country trail that has uphill or downhill grades. In all cases, the step length can be adjusted accordingly to provide optimum Interval Step Training.

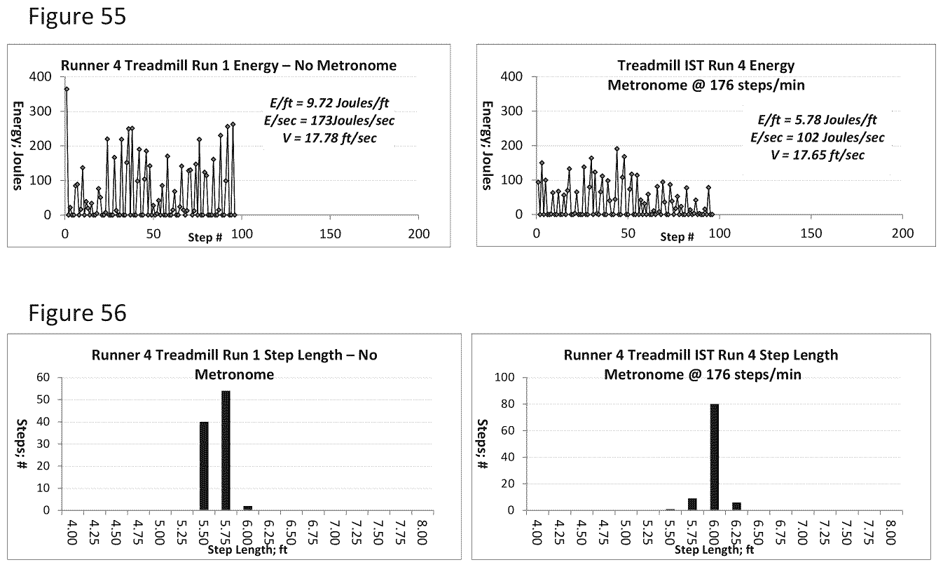

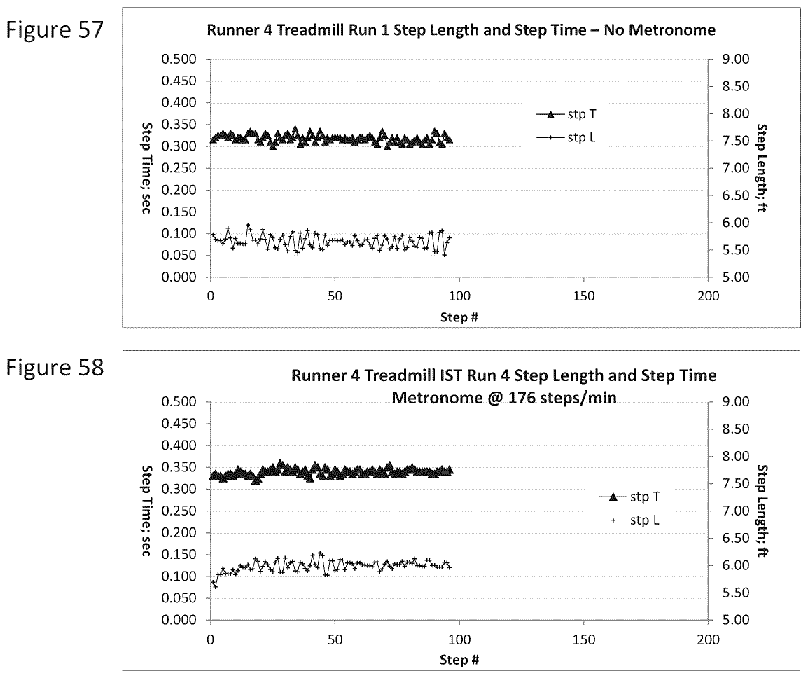

FIG. 53 shows an energy reduction of 18-37% for the IST treadmill runs completed with the metronome turned on to aid the runner in controlling his cadence and step length compared to the runs completed with the metronome turned off. Furthermore, when momentum change (FIG. 54), step energy (FIG. 55), step length distribution (FIG. 56), and step time versus length (FIGS. 57 and 58) are compared, it can be concluded that treadmill running, with a metronome, is viable for IST purposes and can enable runners to run faster and longer at higher speeds by reducing their energy consumption.

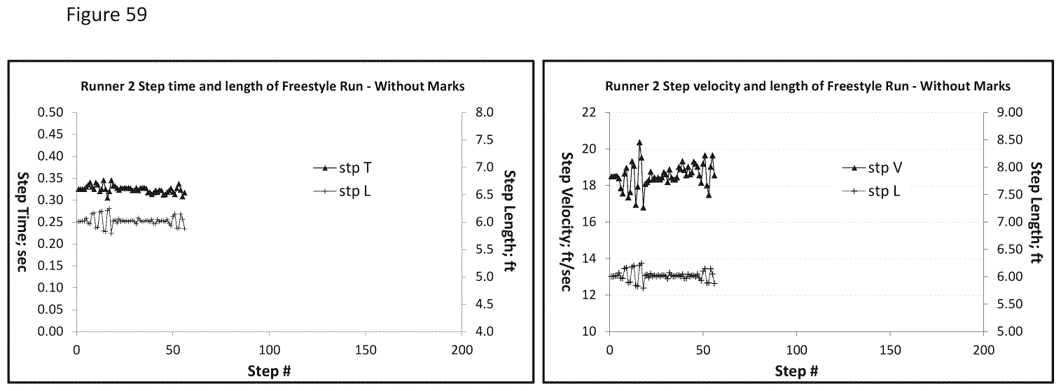

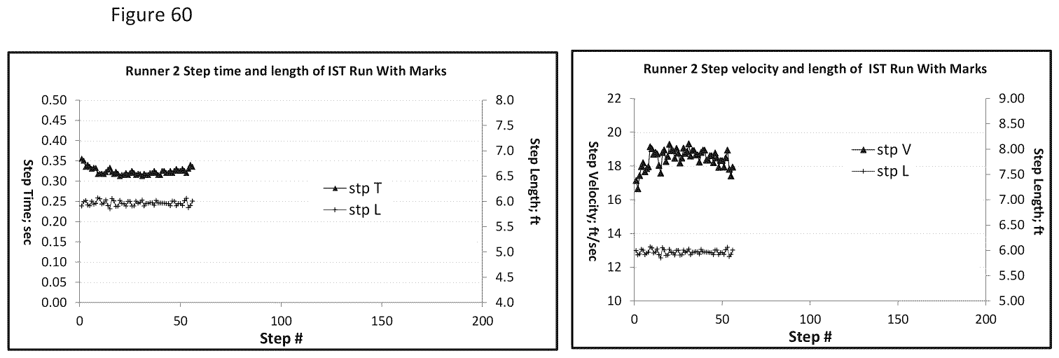

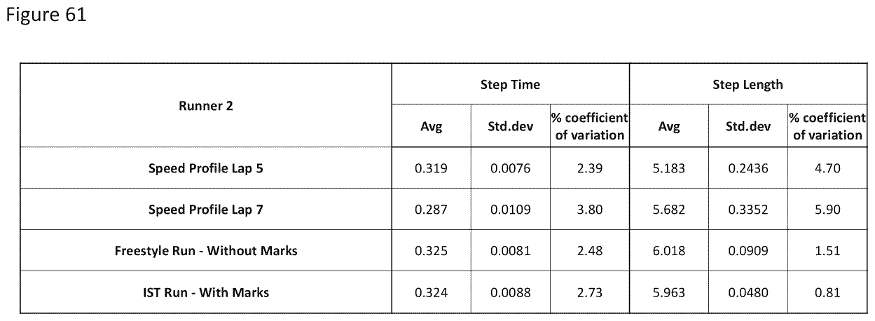

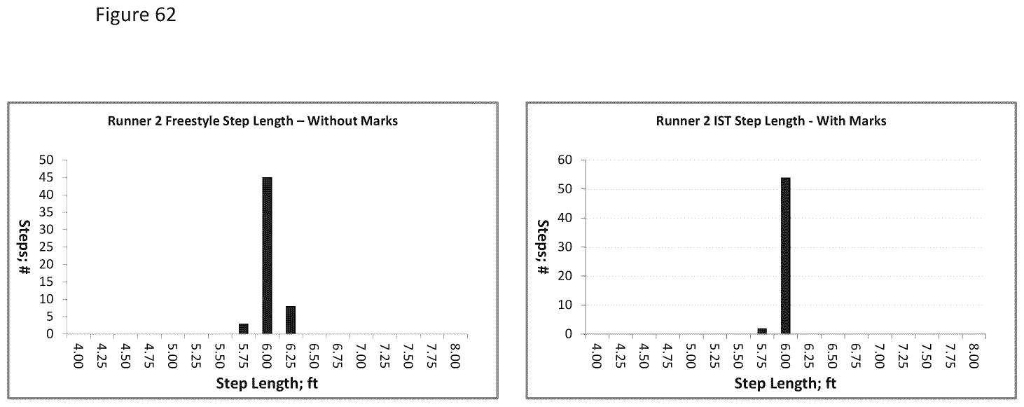

Runner 2 practiced IST two times/week for four weeks. Runner 2's muscle memory was tested with a freestyle run at the end of the fourth week. The freestyle run was done in the same manner as an IST run but instead of running on the marks, Runner 2 moved to an unmarked track lane and ran at a regular race pace (18.54 ft/sec) for the 117 m straight portion. Just like the IST run, he ran the rest of the 400 m track at a recovery pace. He ran two freestyle runs (without marks) and then two IST runs (with the marks). He showed muscle memory of consistent step length on his freestyle run as shown in FIG. 59. When this freestyle run is compared to the IST run shown in FIG. 60, it can be concluded that Runner 2 acquired some muscle memory of consistent step length that mimics his IST run done by stepping on the marks. In FIG. 61, the % coefficient of variation (ratio of the standard deviation to the average) is shown for step time and step length of Runner 2 for speed profile lap 5, lap 7, the freestyle run and IST run. After IST practice, the variability of step length is significantly reduced to 1.51% from profile run lap 7 (5.90%) and lap 5 (4.70%). Coefficient of variation % is even lower (0.81%) for IST run on the marks. So, Runner 2 has room for more improvement regarding step length variation. Also, when the step length distribution of Runner 2's freestyle run and IST run are compared in FIG. 62, muscle memory of consistent step length is observed. Runner 4 also practiced the IST runs for 4 weeks and showed similar muscle memory of consistent step length.

Clause 1—A method for training a runner to reduce energy output including positioning at least three markers, wherein a first marker is a first distance from a second marker, the second marker is a second distance from a third marker, and instructing a runner to run and step on or near the markers while running.

Clause 2—The method of clause 1, wherein the first and second distances are about a step length of the runner.

Clause 3—The method of clauses 1 or 2, wherein the distance between each consecutive marker is about a step length of the runner where the runner minimizes change in the step length between steps.

Clause 4—The method of clauses 1-3, wherein a step-to-step time and/or step-to-step length of the runner is determined.

Clause 5—The method of clauses 1-4, wherein the markers are at least a first indicator, a second indicator, and a third indicator, wherein the first indicator designates that the runner should take shorter steps, the second indicator designates that the runner should take longer steps, and the third indicator designates a correct step, wherein the indicators can emit a sound, a light, or a vibration.

Clause 6—The method of clauses 1-5, further including emitting a sound, light, and/or vibration that corresponds with the moment a runner should step on or near a marker and emitting a sound, light, and/or vibration when a step is landed by the runner.

Clause 7—The method of clause 6, wherein emitting a sound, light, and/or vibration further includes emitting at least a first sound, light, and/or vibration, a second sound, light, and/or vibration, and a third sound, light, and/or vibration, wherein the first sound, light, and/or vibration designates that the runner should take shorter steps, the second sound, light, and/or vibration designates that the runner should take longer steps, and the third sound, light, and/or vibration designates a correct step.

Clause 8—The method of clauses 1-7, wherein a correct step is one in which the step occurs within a correct step length range and at a correct time.

Clause 9—A method for training a runner to reduce energy output including providing a monitoring device including at least one accelerometer, at least one power source, at least one processor, at least one conducting wire, wherein the at least one conducting wire is connected to the accelerometer, wherein the at least one processor receives data from the at least one accelerometer, and processing data from the accelerometer to determine at least one of the following: step length, step time, step velocity, step to step momentum change, and step to step velocity change.

Clause 10—The method of clause 9, wherein the step to step velocity change further comprises determining whether the runner has accelerated or decelerated between steps by comparing an initial pivot velocity to a final pivot velocity.

Clause 11—The method of clauses 9 or 10, further including positioning at least three markers, wherein a first marker is a first distance from a second marker, the second marker is a second distance from a third marker.

Clause 12—The method of clauses 9-11, further including emitting a sound, light, and/or vibration that corresponds to the time when a runner should step on or near a marker, wherein emitting a sound, light, and/or vibration further comprises emitting at least a first sound, light, and/or vibration, a second sound, light, and/or vibration, and a third sound, light, and/or vibration, wherein the first sound, light, and/or vibration designates that the runner should take shorter steps, the second sound, light, and/or vibration designates that the runner should take longer steps, and the third sound, light, and/or vibration designates a correct step.

Clause 13—The method of clauses 9-12, wherein the number of markers corresponds to at least 10 steps by the runner.

Clause 14—The method of clauses 9-13, wherein the monitoring device further comprises a shoe attachment, wherein at least one of the previous running sessions is a speed profile run and wherein the speed profile run includes determining, while a runner runs at least a first speed, at least one of the following: step length, step time, step velocity, step to step momentum change, and step to step velocity change, determining, while a runner runs at least a second speed, at least one of the following: step length, step time, step velocity, step to step momentum change, and step to step velocity change, and wherein the at least a second speed is faster than the at least a first speed.

Clause 15—The method of clauses 9-14, wherein the markers are indicators, wherein the markers are at least a first indicator, a second indicator, and a third indicator, wherein the first indicator designates that the runner should take shorter steps, the second indicator designates that the runner should take longer steps, and the third indicator designates a correct step.

Clause 16—The method of clauses 9-15, wherein the method further includes creating a running timeline by collecting between about 100 and about 1000 data points per second.

Clause 17—The method of clauses 9-16, wherein the runner is running on a treadmill, and a metronome is used for timing.

Clause 18—A monitoring device including at least accelerometer, at least one processor, at least one power source, and at least one conducting wire, wherein the at least one conducting wire is connected to the accelerometer, wherein the at least one processor receives data from the at least one accelerometer.

Clause 19—The monitoring device of clause 18, wherein the monitoring device further includes a shoe attachment, wherein the at least one conducting wire, the accelerometer, the at least one processor, and the power source are connected to the shoe attachment, at least one sensor array, wherein the at least one sensor array comprises at least a first sensor, a second sensor, and a third sensor, wherein the first sensor is located at a heel of the shoe attachment, the second sensor is located at an interior forefoot of the shoe attachment, the third sensor is located at an exterior forefoot of the shoe attachment, conducting material is routed from the first, second, and third sensors to a lateral midfoot region of the shoe attachment, conducting wires connect the conducting material at the lateral midfoot region of the shoe attachment, an electronics housing, a printed circuit board housed within the electronics housing, wherein the accelerometer and the processor are located on the printed circuit board, a conducting wire routed through the electronics housing and connecting to the printed circuit board, and wherein the conducting wires from the shoe attachment are connected to the conducting wires routed through the electronics housing allowing the processor to receive accelerometer data.

Clause 20—The device of clause 18 or 19, wherein the processor can determine at least one of the following: step length, step time, step velocity, step to step momentum change, and step to step velocity change.

Non-limiting aspects have been described, hereinabove. It will be apparent to those skilled in the art that the above methods and apparatuses may incorporate changes and modifications without departing from the general scope of the present subject matter. It is intended to include all such modifications and alterations in so far as they come within the scope of the appended claims or the equivalents thereof.

Having thus described the disclosure, it is now claimed:

I/We claim:

1. A method for training a runner comprising the step of:
positioning at least three markers,
wherein a first marker is a first distance from a second marker, the second marker is a second distance from a third marker; and
instructing a runner to run and step on or near the markers while running.

2. The method of claim 1, wherein the first and second distances are about a step length of the runner.

3. The method of claim 1, wherein the distance between each consecutive marker is about a step length of the runner where the runner minimizes change in the step length between steps.

4. The method of claim 1, wherein a step-to-step time and/or step-to-step length of the runner is determined.

5. The method of claim 4, wherein the markers are at least a first indicator, a second indicator, and a third indicator, wherein the first indicator designates that the runner should take shorter steps, the second indicator designates that the runner should take longer steps, and the third indicator designates a correct step, wherein the indicators can emit a sound, a light, or a vibration.

6. The method of claim 1, further comprising:
emitting a sound, light, and/or vibration that corresponds with the moment a runner should step on or near a marker; and
emitting a sound, light, and/or vibration when a step is landed by the runner.

7. The method of claim 6, wherein emitting a sound, light, and/or vibration further comprises the step of:
emitting at least a first sound, light, and/or vibration, a second sound, light, and/or vibration, and a third sound, light, and/or vibration, wherein the first sound, light, and/or vibration designates that the runner should take shorter steps, the second sound, light, and/or vibration designates that the runner should take longer steps, and the third sound, light, and/or vibration designates a correct step.

8. The method of claim 7, wherein a correct step is one in which the step occurs within a correct step length range and at a correct time.

9. A method for training a runner to reduce energy output comprising the steps of:
providing a monitoring device comprising:
at least one accelerometer;
at least one power source;
at least one processor;
at least one conducting wire, wherein the at least one conducting wire is connected to the accelerometer, wherein the at least one processor receives data from the at least one accelerometer; and
processing data from the accelerometer to determine at least one of the following: step length, step time, step to step momentum change, and step to step velocity change.

10. The method of claim 9, wherein the step to step velocity change further comprises determining whether the runner has accelerated or decelerated between steps by comparing an initial pivot velocity to a final pivot velocity.

11. The method of claim 9, further comprising:
positioning at least three markers, wherein a first marker is a first distance from a second marker, the second marker is a second distance from a third marker.

12. The method of claim 11, further comprising emitting a sound, light, and/or vibration that corresponds to the time when a runner should step on or near a marker, wherein emitting a sound, light, and/or vibration further comprises emitting at least a first sound, light, and/or vibration, a second sound, light, and/or vibration, and a third sound, light, and/or vibration, wherein the first sound, light, and/or vibration designates that the runner should take shorter steps, the second sound, light, and/or vibration designates that the runner should take longer steps, and the third sound, light, and/or vibration designates a correct step.

13. The method of claim 11, wherein the number of markers corresponds to at least 10 steps by the runner.

14. The method of claim 12, wherein the monitoring device further comprises a shoe attachment, wherein at least one of the previous running sessions is a speed profile run and:
wherein the speed profile run comprises:
determining, while a runner runs at least a first speed, at least one of the following: step length, step time, step velocity, step to step momentum change, and step to step velocity change;
determining, while a runner runs at least a second speed, at least one of the following: step length, step time, step velocity, step to step momentum change, and step to step velocity change; and
wherein the at least a second speed is faster than the at least a first speed.

15. The method of claim 11, wherein the markers are indicators, wherein the markers are at least a first indicator, a second indicator, and a third indicator, wherein the first indicator designates that the runner should take shorter steps, the second indicator designates that the runner should take longer steps, and the third indicator designates a correct step.

16. The method of claim 9, wherein the method further comprises the step of:
creating a running timeline by collecting between about 100 and about 1000 data points per second.

17. The method of claim 9, wherein the runner is running on a treadmill, and a metronome is used for timing.

18. A monitoring device comprising:
at least accelerometer;
at least one processor;
at least one power source; and
at least one conducting wire, wherein the at least one conducting wire is connected to the accelerometer, wherein the at least one processor receives data from the at least one accelerometer.

19. The monitoring device of claim 18, wherein the monitoring device further comprises:
a shoe attachment, wherein the at least one conducting wire, the accelerometer, the at least one processor, and the power source are connected to the shoe attachment;
at least one sensor array, wherein the at least one sensor array comprises at least a first sensor, a second sensor, and a third sensor, wherein
the first sensor is located at a heel of the shoe attachment;
the second sensor is located at an interior forefoot of the shoe attachment;
the third sensor is located at an exterior forefoot of the shoe attachment;
conducting material is routed from the first, second, and third sensors to a lateral midfoot region of the shoe attachment;
conducting wires connect the conducting material at the lateral midfoot region of the shoe attachment;
an electronics housing;
a printed circuit board housed within the electronics housing, wherein the accelerometer and the processor are located on the printed circuit board;
a conducting wire routed through the electronics housing and connecting to the printed circuit board; and
wherein the conducting wires from the shoe attachment is connected to the electronics housing allowing the processor to receive shoe attachment and accelerometer data.

20. The device of claim 19, wherein the processor can determine at least one of the following: step length, step time, step velocity, step to step momentum change, and step to step velocity change.
